SEQUENCE LISTING (11 sequences)

SEQ ID NO: 1 - DNA, 1266 bp, Mus musculus
aattctagaactttcctcatgaaaccagctcatctgagaaaacagcaaagtttaaaaaagaaatactatcagtgctgcaaaccaggaaggggaagtgtgtggtttaagttgcatatcccttatcctctggctgctggaggcttgctgaaggctgtatgctgttaatgctaattgtgataggggttttggcctctgactgactcctacctgttagcattaacaggacacaaggcctgttactagcactcacatggaacaaatggccaccgtgggaggatgacaagtccaagagtcaccctgctggatgaacgtagatgtcagactctatcatttaatgtgctagtcataacctggttactaggatagtccactgtaagtgttacgataaatgtcatttaaaagatagatcagcagtatcctaaacaacatctcaacttcaagcccacatgtttattttttatcttgaatggaaagtgaaacttgtatcatttttatttcaaaattatgttcataaccatcttcaatgattcaaccagaatacaaaatgaatgcactaaaaaggacatttctatatttctgtagttaaaattcaggacggccattctcgactgacatctaggattgtctggaatacctcttgtaagacttggaattggcattttttccacattacaatgtattagtcaactttgatttaaaatttgtaactcttgtgttttagtgtaaggaaaaatttagggttagtgttagagtttagggctaggtaaggaaaaactgagtcacactgaatgatttttttaaaatctatgagccagctgttggtagtttactcctttaatcccagcactcgggaggcagagacaggcagatggctgagtcgaaggccaacctggtctacagagtgagctccaggacagccttagctacacagaaaaatgccttatcaaaaaattaagaaaataagatgaagtattaaaaagtgacatgacaaatcattcctgagggctacctatatattcctcacacggtataaatatttaatttaattaatatttaatttcaaatattcacatttgaaatgaaacccaaatctgggttcaagcttactgctttagctgcacagtaaagctgtgtagtaaggagacccacgtttcctacgcatttcttcatgaatgcggatgaaactttacaaggttggtgtgcagctcactggagatgaacaactctttgtaaggtaataaaatcccacagtgatgtcttaaaaaa

SEQ ID NO: 2 - RNA, 65 nt, Mus musculus
cuguuaaugcuaauugugauagggguuuuggccucugacugacuccuaccuguuagcauuaacag

SEQ ID NO: 3 - DNA, 65 bp, Mus musculus
ctgttaatgctaattgtgataggggttttggcctctgactgactcctacctgttagcattaacag

SEQ ID NO: 4 - RNA, 23 nt, Mus musculus
uuaaugcuaauugugauaggggu

SEQ ID NO: 5 - RNA, 21 nt, Mus musculus
cuccuaccuguuagcauuaac

SEQ ID NO: 6 - DNA, 22 bp, Mus musculus
ttaatgctaattgtgatagggg

SEQ ID NO: 7 - DNA, 1421 bp, Homo sapiens
agcggagccccgagccgcccgcagagcaagcgcggggaaccaaggagacgctcctggcactgcagataacttgtctgcatttcaagaacaacctaccagagaccttacctgtcaccttggctctcccacccaatggagatggctctaatggtggcacaaaccaggaaggggaaatctgtggtttaaattctttatgcctcatcctctgagtgctgaaggcttgctgtaggctgtatgctgttaatgctaatcgtgataggggtttttgcctccaactgactcctacatattagcattaacagtgtatgatgcctgttactagcattcacatggaacaaattgctgccgtgggaggatgacaaagaagcatgagtcaccctgctggataaacttagacttcaggctttatcatttttcaatctgttaatcataatctggtcactgggatgttcaaccttaaactaagttttgaaagtaaggttatttaaaagatttatcagtagtatcctaaatgcaaacattttcatttaaatgtcaagcccatgtttgtttttatcattaacagaaaatatattcatgtcattcttaattgcaggttttggcttgttcattataatgttcataaacacctttgattcaactgttagaaatgtgggctaaacacaaatttctataatatttttgtagttaaaaattagaaggactactaacctccagttatatcatggattgtctggcaacgttttttaaaagatttagaaactggtactttcccccaggtaacgattttctgttcaggcaacttcagtttaaaattaatacttttatttgactcttaaagggaaactgaaaggctatgaagctgaatttttttaatgaaatatttttaacagttagcagggtaaataacatctgacagctaatgagatattttttccatacaagataaaaagatttaatcaaaaatttcatatttgaaatgaagtcccaaatctaggttcaagttcaatagcttagccacataatacggttgtgcgagcagagaatctacctttccacttctaagcctgtttcttcctccataaaatggggataatactttacaaggttgttgtgaggcttagatgagatagagaattattccataagataatcaagtgctacattaatgttatagttagattaatccaagaactagtcaccctactttattagagaagagaaaagctaatgatttgatttgcagaatatttaaggtttggatttctatgcagtttttctaaataaccatcacttacaaatatgtaaccaaacgtaattgttagtatatttaatgtaaacttgttttaacaactcttctcaacattttgtccaggttattcactgtaaccaaataaatctcatgagtctttagttgattt

SEQ ID NO: 8 - RNA, 65 nt, Homo sapiens
cuguuaaugcuaaucgugauagggguuuuugccuccaacugacuccuacauauuagcauuaacag

SEQ ID NO: 9 - DNA, 65 bp, Homo sapiens
ctgttaatgctaatcgtgataggggtttttgcctccaactgactcctacatattagcattaacag

SEQ ID NO: 10 - RNA, 23 nt, Homo sapiens
uuaaugcuaaucgugauaggggu

SEQ ID NO: 11 - RNA, 22 nt, Homo sapiens
cuccuacauauuagcauuaaca

(19) United States
(12) Patent Application Publication
(10) Pub. No.: US 2014/0120136 A1
(43) Pub. Date: May 1, 2014

(54) MIR-155 ENHANCEMENT OF CD8+ T CELL IMMUNITY

(71) Applicants: The Babraham Institute, Cambridge (GB); Philadelphia Health & Education Corporation d/b/a Drexel University College of Medicine, Philadelphia, PA (US)

(72) Inventors: Peter D. Katsikis, Merion Station, PA (US); Donald T. Gracias, Philadelphia, PA (US); Martin Turner, Cambridge (GB)

(73) Assignees: The Babraham Institute, Cambridge (GB); Philadelphia Health & Education Corporation d/b/a Drexel University College of Medicine, Philadelphia, PA (US)

(21) Appl. No.: 14/051,895

(22) Filed: Oct. 11, 2013

Related U.S. Application Data
(60) Provisional application No. 61/712,882, filed on Oct. 12, 2012.

(51) Int. Cl. C12N 15/113

(52) U.S. Cl. CPC C12N 15/113
USPC 424/204.1; 424/184.1; 424/277.1; 424/234.1

(57) ABSTRACT

The present invention provides novel methods of enhancing CD8+ T cell mediated immunity (also referred to as “CD8+ T cell immunity”) in a patient having a diseased state. In particular, the present invention provides for the enhanced expression of miR-155 in a population of patient specific T cells through the introduction of a nucleic acid molecule encoding a miR-155 transcript or a nucleic acid molecule encoding a chimeric antigen receptor and a miR-155 transcript into those cells, followed by the reintroduction of the T cells into the patient. The present invention also provides methods of enhancing the expansion of these T cells relative to control cells. Increased expansion of CD8+ T cells following enhanced miR-155 expression is directly related to enhanced CD8+ T cell immunity. The present invention further provides methods of enhancing anti-cancer immunity in a patient through the increased expression of miR-155 in patient specific T cells.

### CROSS REFERENCE TO RELATED APPLICATIONS

This patent application claims the benefit under 35 U.S.C. §119(e) to U.S. provisional application No. 61/712,882, filed Oct. 12, 2012, entitled miR-155 Enhancement of CD8+T Cell Immunity, the contents of which is incorporated by reference herein in its entirety.

### GOVERNMENT RIGHTS

The work presented here was supported by the Institute of Molecular Medicine and Infectious Disease of Drexel University College of Medicine, NIH grants R01 AI66215 and R01 AI46719 to Dr. Peter Katsikis and funding from the Biotechnology and Biological Sciences Research Council and Medical Research Council to Dr. Martin Turner. The Government has certain rights in the herein disclosed subject matter.

### TECHNICAL FIELD

This disclosure provides methods of utilizing miR-155 to enhance the T cell mediated immunity of a subject having a disease state. Also disclosed herein are methods of increasing anti-tumor immunity in a subject through the ex vivo transduction of a population of the subject's T cells with miR-155. Additionally, disclosed herein are methods of increasing the expansion of T cells in a subject having a disease state.

### BACKGROUND

MicroRNAs (miRs) are small non-coding RNAs, approximately 22 nucleotides long, which play a key role in post-transcriptional gene modulation in mammals (Lodish et al., Nature Reviews (2008) 8:120-130; Lewis et al., Cell (2005) 120:15-20; Miranda et al., Cell (2006) 126:1203-1217; Friedman et al., Genome Res. (2009) 19:92-105). Currently, over 1000 miRs have been identified in humans. (Di Leva et al., Upsala Journal of Medical Sciences (2012) 117: 202-216). MiRs play a role in numerous biological processes including cell growth and differentiation, cell cycle control, apoptosis, stress response, and cancer. (Di Leva et al., Upsala Journal of Medical Sciences (2012) 117: 202-216).

One miR that has been shown to play a role in numerous biological processes is miR-155. MiR-155 is classified as an onco-miR, playing a role in initiating or accelerating cancer (Di Leva et al., Upsala Journal of Medical Sciences (2012) 117:202-16). Overexpression of miR-155 in mouse hematopoietic cells induces malignancy (Costinean et al., PNAS (2006) 103:7024-29) and miR-155 is overexpressed in bone marrow of humans with acute myeloid leukemia (O'Connell et al., J Exp Med. (2008) 205:585-94).

MiR-155 also has an emerging role in regulating immune responses. MiR-155 is required for proper immune response to Salmonella typhimurinum (Rodriguez et al., Science (2007) 316:608-11), H. pylori (Oertili et al., J Immunol. (2011) 187:3578-86), influenza virus and Listeria monocytogenes (Stelekati et al., J Immunol. (2009) 182, 90.23). MiR-155 expression is induced in macrophages following RNA virus infection (Wang et al., J Immunol. (2010) 185:6226-33) and miR-155 is upregulated in response to T cell activation (Haasch et al., Cell. Immunol. (2002) 217:78-86). Additionally, miR-155 is upregulated upon CD8+ T cell activation while the in vivo response of CD8+ T cells is reduced following miR-155 deficiency (Stelekati et al., J Immunol. (2009) 182, 90.23).

### SUMMARY OF THE INVENTION

The present invention relates to methods of utilizing miR-155 to enhance T cell mediated immunity in a subject having a disease state. More specifically, the inventors have discovered that, by using ex vivo techniques, introducing a nucleic acid molecule encoding a miR-155 transcript into a population of subject specific T cells results in the enhanced expansion of those cells corresponding to an enhanced immune response.

In one embodiment, the invention is directed to a method of enhancing CD8+ T cell mediated immunity in a subject having a disease state comprising:
a) isolating a population of CD8+ T cells from the subject;
b) introducing a nucleic acid molecule encoding a miR-155 transcript into the isolated CD8+ T cells; and
c) reintroducing the CD8+ T cells into the subject.

In another embodiment of the present invention, the invention is directed to a method of increasing T cell mediated immunity in a subject having a disease state comprising:
a) isolating a population of the subject's T cells;
b) introducing a nucleic acid molecule encoding a chimeric antigen receptor and a miR-155 transcript into the isolated T cells; and
c) reintroducing the T cells into said subject.

In another embodiment of the present invention, the invention is directed to a method of increasing T cell mediated immunity in a subject having a disease state comprising:
a) isolating a population of the subject's T cells;
b) introducing a nucleic acid molecule encoding a chimeric antigen receptor into the T cells;
c) additionally introducing a nucleic acid molecule encoding a miR-155 transcript into the T cells; and
d) reintroducing the T cells into said subject.

The present invention also provides methods of enhancing the expansion of T cells relative to control cells.

The present invention further provides methods of enhancing anti-cancer immunity in a patient through the increased expression of miR-155 in patient specific T cells.

### BRIEF DESCRIPTION OF THE DRAWINGS

The summary, as well as the following detailed description, is further understood when read in conjugation with the appended drawings. For the purpose of illustrating the invention, there are shown in the drawings exemplary embodiments for the invention; however, the invention is not limited to the specific methods and composition disclosed. In addition, the drawings are not necessarily drawn to scale.

FIG. 1, comprising FIG. 1A-1B, illustrates miR-155 expression in CD8+ T cells. (A) miR-155 is highly upregulated with in vitro activation of CD8+ T cells. Sorted splenic CD8+ T cells from wild-type C57BL/6 mice were stimulated in vitro with anti-CD3/anti-CD28 antibodies for 1, 3 and 5 days and miR-155 expression was quantified by RT-PCR. Graph shows fold increase of miR-155 expression over sorted unstimulated CD8+ T cells. Bars represent mean and standard errors of 5 animals per group tested in 3 independent experiments. (B) miR-155 is expressed in vivo in primary effector and effector memory CD8+ T cells. Donor day 10 lung effector CD44+CD62L− CD8+ T cells and donor day 60 splenic effector memory CD44+CD62L− or central memory CD44+CD62L+ CD8+ T cells were sorted from congenic animals that had received OT-I adoptive transfers and been infected with WSNOVA influenza virus. Naïve CD44−CD62L+ CD8+ T cells were sorted from spleens of naïve OT-I mice. MiR-155 expression was quantified by RT-PCR. Graph depicts fold increase of miR-155 over naïve CD8+ T cells. Bars represent mean and SEM from 3-5 mice/groups and 2 independent experiments. All values were normalized to 18S rRNA expression.

FIG. 2, comprising FIG. 2A-2K, illustrates the requirement of miR-155 for CD8+ T cell responses. Reduced CD8+ T cell responses (A) and (B) and decreased viral clearance (C) in A/PR/8/34 influenza virus infected miR-155−/− mice. (A) Representative FACS plots and (B) numbers of day 10 lung NP(366-374)-specific CD8+ T cells are shown. (C) Day 10 lung viral loads of wild-type C57BL/6 and miR-155−/− mice. TCID50 per 100 mg of lung tissue shown. Data are from 3 independent experiments and n=8-10 mice per group. *P<0.002 and **P<0.05 (Student's t-test). (D), (E), (F) Impaired response of miR-155−/− CD8+ T cells to influenza virus infection. Donor CD8+ T cells shown from congenic animals that had received adoptive transfers of wild type or miR-155−/− CD8+ T cells and been infected with WSN-OVA influenza virus. Representative FACS plots of lung (D), and numbers (E) of donor OVA(257-264)-specific CD8+ T cells in lungs, mediastinal LN (MLN) and spleens shown. Horizontal lines depict means of 4 independent experiments (n=11 per group). *P<0.001 (Student's t-test for lungs and spleens, Mann-Whitney U test for MLN). (F) Numbers of lung donor OVA(257-264)-specific CD8+ T cells for days 7, 10 and 14 post-infection shown (n=4 per group). (G) and (H) Reduced response of miR-155 deficient CD8+ T cells against Listeria monocytogenes infection. Donor CD8+ T cells shown from congenic animals that had received adoptive transfers of wild type or miR-155−/− CD8+ T cells and been infected with L.m.-OVA. Representative FACS plots of spleens (G) and numbers (H) of day 7 post-infection donor IFN-γ+ OVA(257-264) peptide-stimulated CD8+ T cells in spleens and mesenteric LN are depicted. Horizontal lines depict means of two independent experiments (n=7). *P<0.001 (Student's t-test). (I) and (J) miR-155 deficiency impairs CD8+ T cell memory generation. Representative FACS plots of spleens (I) and numbers in spleens (J) of day 60 donor memory CD8+ T cells from congenic animals that had received adoptive transfers of wild type or miR-155−/− CD8+ T cells and been infected with WSN-OVA influenza virus. Means and SEM of 2 independent experiments (n=5 per group). (K) Increased expansion of miR-155 overexpressing CD8+ T cells. Fold expansion shown of day 10 lung donor OT-I cells in animals that had received adoptive transfers of retrovirally transduced OT-I cells and were infected with WSN-OVA virus. Bars show mean±SEM of 3 independent experiments (n=8-10 per group). For (j) and (k), *P<0.05 (Mann-Whitney U test).

FIG. 3, comprising FIG. 3A-3D, illustrates miR-155 deficiency impairing CD8+ T cell proliferation. (A) and (B) Purified and CFSE-labeled CD8+ T cells stimulated with OVA(257-264)-pulsed irradiated splenocytes for 4 days. (A) Representative histogram plot of CFSE dilution showing reduced proliferation of miR-155−/−OT-I CD8+ T cells compared to OT-I CD8+ T cells. (B) Bar graph shows mean±SEM of absolute number of live CD8+ T cells. Data from 5 independent experiments (n=5 per group). *P<0.02 (Student's t-test). (C) Reduced 3H-Thymidine incorporation by purified miR-155−/− CD8+ T cells stimulated with anti-CD3 antibody+IL-2 for 5 days. Bars show mean±SEM of triplicates of representative experiment of 4 performed. (D) TCR-stimulated intracellular calcium flux is not affected in miR-155. Representative histogram of 3 experiments performed shown. Arrows indicate stimulation time point.

FIG. 4 illustrates the reduction in miR-155−/−CD8+ T cell memory in the MLN and lungs. Day 60 memory miR-155−/−OT-I and wild-type OT-I in the MLN and lungs of mice that received adoptive transfers of wild type and miR-155−/− CD8+ T cells and were infected with WSN-OVA influenza virus. Memory in spleens is shown in FIG. 1. Bars shows mean±SEM and are from 2 independent experiments (n=5).

FIG. 5 illustrates miR-155 overexpressing CD8+ T cells inhibiting AE17.OVA tumor growth. Mice were injected in the flank with mesothelioma tumor AE17 expressing OVA (AE17.OVA) and 10 days later 2×10⁵ retrovirally transduced OT-I cells were injected intravenously and tumor growth was measured daily. OT-I cells were retrovirally transduced with a retrovirus expressing miR-155 or a control vector. P values shown are for comparisons between OT-I control vector and OT-I miR-155 overexpression. N=5 mice per group.

FIG. 6, comprising FIGS. 6A-6B, illustrates the increased in vivo anti-viral CD8+ T cell expansion upon miR-155 overexpression. Mice were injected intravenously with 1×10⁴ retrovirally transduced OT-I cells and the infected with OVA expressing influenza virus (WSN-OVA). Mice were harvested on day 10. OT-I cells were retrovirally transduced with a retrovirus expressing miR-155 or a control vector. (A) Representative FACS plots showing day 10 OVA-specific CD8+ T cell responses in lungs. (B) Pooled data showing the numbers of lung OVA-specific CD8+ T cell response on day 10 of infection. N=5 mice per group.

### DETAILED DESCRIPTION OF THE INVENTION

The present invention may be understood more readily by reference to the following detailed description taken in connection with the accompanying figures and examples, which form a part of this disclosure. It is to be understood that this invention is not limited to the specific methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

It is to be understood that the invention is not limited to the particular embodiments described below, as variations of the described embodiments may fall within the enclosed claims. Also, the terminology used to describe the enclosed embodiments is not intended to be limiting. The scope of the present invention is established by the claims.

MiR-155 has an emerging role in the regulation of immune responses. Disclosed herein are methods of utilizing miR-155 to enhance the T cell mediated immunity of a subject having a disease state. Also disclosed herein are methods of increasing anti-tumor immunity in a subject through the ex vivo transduction of a population of the subject's T cells with miR-155. Additionally, disclosed herein are methods of increasing the expansion of T cells in a subject having a disease state.

Use of miR-155 to Enhance T Cell Mediated Immunity.

As described above, this invention provides a method of utilizing miR-155 to enhance T cell mediated immunity in a subject. In one embodiment of the present invention, a population of CD8+ T cells is isolated from a subject, a nucleic acid molecule encoding a miR-155 transcript is introduced into the CD8+ T cells, and the cells are reintroduced into the subject.

MicroRNAs (miRs) are small non-coding RNAs, approximately 22 nucleotides long, which play a key role in post-transcriptional gene modulation in mammals. Several rounds of processing are required to form the mature miR. The DNA encoding the miR is transcribed by RNA polymerase II to form a primary miR (pri-miR). The pri-miR is processed to form a pre-miR (stem-loop), which is transported out of the nucleus and further processed to form the mature miR. As used herein, miR-155 refers to the miR-155 transcript encoded by human chromosome 21, mouse chromosome 16, or any homologous miR transcript from another species. Thus, in some embodiments of the present invention miR-155 is a human miR. In other embodiments the miR-155 is a mouse miR. In other embodiments miR-155 is from another species. As used herein, a “nucleic acid molecule encoding miR-155” refers to the BIC gene or a portion thereof that encodes a miR-155 transcript. “Nucleic acid molecule” refers to DNA and RNA. Thus, in some embodiments, the nucleic acid molecule introduced into the T cells is the DNA of the full length BIC gene. In other embodiments, the nucleic acid molecule introduced into the T cells is the DNA of a portion of the BIC gene, which encodes at least the miR-155 transcript. Nucleic acid molecule also includes unprocessed, partially processed, and mature RNA. Thus, in some embodiments, a pri-miR encoding miR-155 is introduced into the T cells. In other embodiments, a pre-miR is introduced into the T cells. In further embodiments, miR-155 in the mature, processed form is introduced into the T cells. In some embodiments, the sense strand (miR-155-5p) is introduced into the T cells. In other embodiments, the antisense strand (miR-155-3p) is introduced into the T cells.

Exemplary miR-155 sequences include, but are not limited to, those listed in Table 1.

TABLE 1

| | Sequence | Accession Number |
|---|---|---|
| Mouse BIC non- | AATTCTAGAACTTTCCTCATGAAACCAGCT | AY096003 |
| coding mRNA | CATCTGAGAAAACAGCAAAGTTTAAAAAA | |
| | GAAATACTATCAGTGCTGCAAACCAGGAAG | |
| | GGGAAGTGTGTGGTTTAAGTTGCATATCCC | |
| | TTATCCTCTGGCTGCTGGAGGCTTGCTGAA | |
| | GGCTGTATGCTGTTAATGCTAATTGTGATA | |
| | GGGGTTTTGGCCTCTGACTGACTCCTACCTG | |
| | TTAGCATTAACAGGACACAAGGCCTGTTAC | |
| | TAGCACTCACATGGAACAAATGGCCACCGT | |
| | GGGAGGATGACAAGTCCAAGAGTCACCCTG | |
| | CTGGATGAACGTAGATGTCAGACTCTATCA | |
| | TTTAATGTGCTAGTCATAACCTGGTTACTAG | |
| | GATAGTCCACTGTAAGTGTTACGATAAATG | |
| | TCATTTAAAAGATAGATCAGCAGTATCCTA | |
| | AACAACATCTCAACTTCAAGCCCACATGTT | |
| | TATTTTTTATCTTGAATGGAAAGTGAAACTT | |
| | GTATCATTTTTATTTCAAAATTATGTTCATA | |
| | ACCATCTTCAATGATTCAACCAGAATACAA | |
| | AATGAATGCACTAAAAAGGACATTTCTATA | |
| | TTTCTGTAGTTAAAATTCAGGACGGCCATTC | |
| | TCGACTGACATCTAGGATTGTCTGGAATAC | |
| | CTCTTGTAAGACTTGGAATTGGCATTTTTTC | |
| | CACATTACAATGTATTAGTCAACTTTGATTT | |
| | AAAATTTGTAACTCTTGTGTTTTAGTGTAAG | |
| | GAAAAATTTAGGGTTAGTGTTAGAGTTTAG | |
| | GGCTAGGTAAGGAAAAACTGAGTCACACTG | |
| | AATGATTTTTTTAAAATCTATGAGCCAGCTG | |
| | TTGGTAGTTTACTCCTTTAATCCCAGCACTC | |
| | GGGAGGCAGAGACAGGCAGATGGCTGAGT | |
| | CGAAGGCCAACCTGGTCTACAGAGTGAGCT | |
| | CCAGGACAGCCTTAGCTACACAGAAAAATG | |
| | CCTTATCAAAAAATTAAGAAAATAAGATGA | |
| | AGTATTAAAAAGTGACATGACAAATCATTC | |
| | CTGAGGGCTACCTATATATTCCTCACACGG | |
| | TATAAATATTTAATTTAATTAATATTTAATT | |
| | TCAAATATTCACATTTGAAATGAAACCCAA | |
| | ATCTGGGTTCAAGCTTACTGCTTTAGCTGCA | |
| | CAGTAAAGCTGTGTAGTAAGGAGACCCACG | |
| | TTTCCTACGCATTTCTTCATGAATGCGGATG | |
| | AAACTTTACAAGGTTGGTGTGCAGCTCACT | |
| | GGAGATGAACAACTCTTTGTAAGGTAATAA | |
| | AATCCCACAGTGATGTCTTAAAAAA | |
| Mouse miR-155 | CUGUUAAUGCUAAUUGUGAUAGGGGUUUU | MI0000177 |
| stem-loop | GGCCUCUGACUGACUCCUACCUGUUAGCA | |
| | UUAACAG | |
| Mouse miR-155 | CTGTTAATGCTAATTGTGATAGGGGTTTTGG | NR_029565 |
| stem-loop | CCTCTGACTGACTCCTACCTGTTAGCATTAA | |
| | CAG | |
| Mouse mature | UUAAUGCUAAUUGUGAUAGGGGU | MIMAT0000165 |
| miR-155-5p | | |
| Mouse mature | CUCCUACCUGUUAGCAUUAAC | MIMAT0016993 |
| miR-155-3p | | |
| Mouse miR-155 | TTAATGCTAATTGTGATAGGGG | AJ459767 |
| Human BIC | AGCGGAGCCCCGAGCCGCCCGCAGAGCAA | AF402776 |
| noncoding mRNA | GCGCGGGGAACCAAGGAGACGCTCCTGGC | |
| | ACTGCAGATAACTTGTCTGCATTTCAAGAA | |
| | CAACCTACCAGAGACCTTACCTGTCACCTT | |
| | GGCTCTCCCACCCAATGGAGATGGCTCTAA | |
| | TGGTGGCACAAACCAGGAAGGGGAAATCT | |
| | GTGGTTTAAATTCTTTATGCCTCATCCTCTG | |
| | AGTGCTGAAGGCTTGCTGTAGGCTGTATGC | |
| | TGTTAATGCTAATCGTGATAGGGGTTTTTGC | |
| | CTCCAACTGACTCCTACATATTAGCATTAAC | |
| | AGTGTATGATGCCTGTTACTAGCATTCACAT | |
| | GGAACAAATTGCTGCCGTGGGAGGATGACA | |
| | AAGAAGCATGAGTCACCCTGCTGGATAAAC | |
| | TTAGACTTCAGGCTTTATCATTTTTCAATCT | |
| | GTTAATCATAATCTGGTCACTGGGATGTTC | |
| | AACCTTAAACTAAGTTTTGAAAGTAAGGTT | |
| | ATTTAAAAGATTTATCAGTAGTATCCTAAA | |
| | TGCAAACATTTTCATTTAAATGTCAAGCCC | |
| | ATGTTTGTTTTTATCATTAACAGAAAATATA | |
| | TTCATGTCATTCTTAATTGCAGGTTTTGGCT | |
| | TGTTCATTATAATGTTCATAAACACCTTTGA | |
| | TTCAACTGTTAGAAATGTGGGCTAAACACA | |
| | AATTTCTATAATATTTTTGTAGTTAAAAATT | |
| | AGAAGGACTACTAACCTCCAGTTATATCAT | |
| | GGATTGTCTGGCAACGTTTTTTAAAAGATTT | |
| | AGAAACTGGTACTTTCCCCCAGGTAACGAT | |
| | TTTCTGTTCAGGCAACTTCAGTTTAAAATTA | |
| | ATACTTTTATTTGACTCTTAAAGGGAAACTG | |
| | AAAGGCTATGAAGCTGAATTTTTTTAATGA | |
| | AATATTTTTAACAGTTAGCAGGGTAAATAA | |
| | CATCTGACAGCTAATGAGATATTTTTTCCAT | |
| | ACAAGATAAAAAGATTTAATCAAAAATTTC | |
| | ATATTTGAAATGAAGTCCCAAATCTAGGTT | |
| | CAAGTTCAATAGCTTAGCCACATAATACGG | |
| | TTGTGCGAGCAGAGAATCTACCTTTCCACTT | |
| | CTAAGCCTGTTTCTTCCTCCATAAAATGGGG | |
| | ATAATACTTTACAAGGTTGTTGTGAGGCTT | |
| | AGATGAGATAGAGAATTATTCCATAAGATA | |
| | ATCAAGTGCTACATTAATGTTATAGTTAGA | |
| | TTAATCCAAGAACTAGTCACCCTACTTTATT | |
| | AGAGAAGAGAAAAGCTAATGATTTGATTTG | |
| | CAGAATATTTAAGGTTTGGATTTCTATGCA | |
| | GTTTTTCTAAATAACCATCACTTACAAATAT | |
| | GTAACCAAACGTAATTGTTAGTATATTTAA | |
| | TGTAAACTTGTTTTAACAACTCTTCTCAACA | |
| | TTTTGTCCAGGTTATTCACTGTAACCAAATA | |
| | AATCTCATGAGTCTTTAGTTGATTT | |
| Human miR-155 | CUGUUAAUGCUAAUCGUGAUAGGGGUUUU | MI0000681 |
| stem-loop | UGCCUCCAACUGACUCCUACAUAUUAGCA | |
| | UUAACAG | |
| Human miR-155 | CTGTTAATGCTAATCGTGATAGGGGTTTTTG | NR_030784 |
| stem-loop | CCTCCAACTGACTCCTACATATTAGCATTAA | |
| | CAG | |
| Human mature | UUAAUGCUAAUCGUGAUAGGGGU | MIMAT0000646 |
| miR-155-5p | | |
| Human mature | CUCCUACAUAUUAGCAUUAACA | MIMAT0004658 |
| miR-155-3p | | |

“Adoptive cell transfer” as used herein is the passive transfer of cells into a host. Adoptive cell transfers can be autologous or heterologous transfers. In autologous transfers, the cells are isolated from an individual and then re-infused into the same subject. In heterologous transfers, the cells are isolated from one individual and infused into a different subject. Cells isolated from a host can be manipulated and treated to enhance function or expand their numbers before infusion into a host. Thus, by way of example, CD8+ or CD4+ T cells can be expanded with anti-CD3+anti-CD28 antibodies in vitro either before or after introduction of the nucleic acid molecule into those cells.

The term “subject” as used herein is intended to mean any animal, in particular mammals. Although the enhancement of CD8+ T cell mediated immunity in mice is exemplified herein, any type of mammal can be treated using the present invention. Thus, the method of the invention is applicable to human and nonhuman animals, although it is most preferably used with mice and humans, and most preferably with humans. “Subject” and “patient” are used interchangeably herein.

T cells may be isolated from the subject's blood, lymph nodes, tumor or site of infection. Isolation and purification of T cells from the blood, lymph nodes and tumor can be performed by a variety of techniques known in the art. These methods include but are not limited to: leukapheresis; density centrifugation; panning; positive and negative selection using magnetic beads, magnetic microparticles and magnetic nanoparticles (using directly antibody coated beads and particles, or using antibodies to coat cells and then beads and particles with a secondary reagent to bind antibodies); positive and negative selection using red blood cells, beads, microparticles and nanoparticles and density centrifugation (using directly antibody coated beads and particles, or using antibodies to coat cells and then beads and particles with a secondary reagent to bind antibodies); Fluorescence Activated Cell Sorting (FACS); and laser capture microdissection. These methods can be used alone or in combinations to improve purity and yield.

Ex vivo as used herein refers to experimentation or procedures performed outside of the subject.

A nucleic acid molecule encoding a miR-155 transcript can be introduced, ex vivo, into isolated T cells using a wide variety of techniques well known in the art. In some embodiments, the nucleic acid molecule is introduced into the cells by transduction. Transduction involves the use of viruses and viral vectors to introduce the nucleic acid molecule into the isolated cells. Viral vectors include, but are not limited to, lentivirus, retrovirus and adenovirus vectors. Thus, in one example of the present invention, CD8+ T cells isolated from the subject are transduced with a miR-155-expressing MIGR1 retroviral vector. In other embodiments, the nucleic acid molecule is introduced into the cells by transfection. Transfection procedures are well known in the art and include the use of liposomes, calcium phosphate, electroporation, dendrimers, cyclodextrin, polymers, nanoparticles and nanofibers. Transfections include the use of non-viral vectors such as plasmid vectors. Thus, in some embodiments, the nucleic acid molecule encoding a miR-155 transcript is introduced into the isolated T cells from the subject by transfection using a plasmid vector.

T cells can be reintroduced into the patient using a wide variety of techniques known in the art. In some embodiments, T cells are transferred intravenously into the subject. In other embodiments, T cells are injected into a tumor or site of infection by methods including, but not limited to, intratumoral, subcutaneous, intraperitoneal, intracranial, intradermal and intra-CSF injections.

In some embodiments of the present invention, T cells are isolated from the subject, a nucleic acid molecule encoding a chimeric antigen receptor (CAR) (also referred to as “chimeric immunoreceptor” or “chimeric T cell receptor”) and a miR-155 transcript are introduced into the T cells, and the T cells are reintroduced into the subject.

Methods of engineering CAR T cells and their use in creating genetically modified cells to stimulate T cell mediated immune response is known in the art (see WO 2012/079000; WO 2010/025177; WO 2006/060878; U.S. Pat. No. 6,410,319). CARs are composed of an extracellular antigen binding domain, a transmembrane domain, and an intracellular/cytoplasmic domain. The extracellular antigen binding domain directs the specificity of the T cell expressing the CAR, introducing a novel specificity or redirecting the specificity of the cell in which it is expressed. The intracellular/cytoplasmic domain of the CAR is responsible for at least one effector function of the CAR expressing cell. Such effector function can be derived from, for example, CD3-zeta by itself or in combination with other costimulatory signaling molecules such as CD28 or CD137 (4-1BB). Incorporating miR-155 into engineered receptors such as CARs is contemplated to potentiate the anti-tumor or anti-infective activity of cells containing those receptors.

The nucleic acid molecule encoding a miR-155 transcript can be incorporated into the intracellular domain of the CAR sequence such that it is expressed intracellularly upon introduction into the T cell. In some embodiments, the intracellular domain contains miR-155 by itself. In other embodiments, the intracellular domain contains miR-155 in combination with the cytoplasmic domain of the T cell receptor or other antigen binding molecule. For example, the intracellular domain can contain miR-155 in combination with known costimulatory signaling molecules. In yet another examples, the intracellular domain can contain miR-155 together with the T cell receptor cytoplasmic domain and costimulatory signaling molecules. Thus, by way of example, a nucleic acid molecule encoding a CAR comprising an extracellular antigen binding domain, a transmembrane domain, and an intracellular domain consisting of miR-155, CD3-zeta and 4-1BB is introduced into a population of patient T cells, which are then reintroduced into the patient resulting in enhanced T cell mediated immunity.

The present invention provides an improvement over the prior art CAR technology. Engineered CAR T cells produce increased immune responses in the absence of enhanced T cell proliferation/expansion (WO 2006/060878; WO 2010/025177). By engineering miR-155 into the intracellular tail of such chimeric receptors, one can enhance the ability of T cells expressing such receptors to expand or function and thus further enhance their anti-tumor or anti-infective activity. Thus, the present invention is advantageous over the prior art as it combines the enhanced expansion effect of miR-155 transduced T cells with the enhanced immune activity of CART cells. Specifically, addition of the miR-155 sequence to the intracellular domain of the CAR will enhance the immune response of T cells expressing these receptors, thus providing enhanced T cell mediated immunity in a subject with a disease state.

In a further embodiments of the present invention, T cells are isolated from the subject, a nucleic acid molecule encoding a CAR is introduced into the T cells, additionally a nucleic acid molecule encoding a miR-155 transcript is introduced into the T cells, and the T cells are reintroduced into the subject. “Additionally” as used herein is not intended to require a temporal order in which the nucleic acid molecule is added into the T cells. Thus, a plurality of vectors encoding nucleic acid molecules, comprising at least a nucleic acid molecule encoding a CAR and a nucleic acid molecule encoding a miR-155 transcript, can be introduced into the isolated T cells, simultaneously or subsequent to each other. In some embodiments, a vector comprising a nucleic acid molecule encoding a CAR can be first introduced into the T cells, followed by a vector comprising a nucleic acid molecule encoding a miR-155 transcript. In other embodiments, a vector comprising a nucleic acid molecule encoding a miR-155 transcript can be first introduced into the T cells followed by a vector comprising a nucleic acid molecule encoding a CAR. In other embodiments, a vector comprising a nucleic acid molecule encoding a miR-155 transcript can be introduced into the T cells simultaneously with a vector comprising a nucleic acid molecule encoding a CAR. By introducing a plurality of vectors comprising nucleic acid molecules into T cells, patient specific T cells expressing at least CAR and miR-155 can be generated.

In some embodiments, the population of T cells isolated from the subject can comprise both CD4+ T cells and CD8+ T cells. In other embodiments, the population of T cells isolated from the subject can comprise CD8+ T cells. In other embodiments, the population of T cells isolated from the subject can comprise CD4+ T cells.

Use of miR-155 for the Enhancement of Anti-Cancer Immunity.

MiR-155 is a known onco-miR, inducing tumor growth, aggressiveness, and resistance to chemotherapy when expressed ectopically in vitro or in vivo (Di Leva et al., Upsala Journal of Medical Sciences (2012), 117: 202-216). Additionally, miR-155 transgenic mice develop preleukemic pre-B cell proliferation and by 6 months of age develop high-grade B cell neoplasm (Costinean et al., PNAS (2006) 103:7024-29). While these studies highlight the ability of miR-155 to contribute to the formation of cancer, the present invention provides the unexpected result that enhancement of miR-155 expression in a population of patient-specific CD8+ T cells enhances the patient's immune response.

Thus, methods of providing anti-cancer immunity in a subject are disclosed. In some embodiments, a nucleic acid molecule encoding a miR-155 transcript can be introduced into subject-specific CD8+ T cells. In other embodiments, a nucleic acid molecule encoding a CAR and a miR-155 transcript can be introduced into subject-specific T cells. In other embodiments, a nucleic acid molecule encoding a CAR and a nucleic acid molecule encoding a miR-155 transcript can be introduced into subject-specific T cells.

Cancers contemplated to be amenable to treatment with the methods disclosed herein include solid tumors, hematologic cancers, and other oncogenic malignancies. For solid tumors, the present invention provides a method of isolating a population of subject-specific tumor-infiltrating T cells, introducing a nucleic acid molecule into those T cells, and reintroducing the T cells into the subject.

For hematologic cancers, the present invention provides a method of isolating a population of subject-specific T cells from the blood, introducing a nucleic acid molecule into those cells, and reintroducing the T cells into the subject. It is understood by those with skill in the art that hematologic cancers are those originating in blood forming tissues or in the cells of the immune system.

The use of miRs for the treatment of cancer is known in the art (see for example U.S. Pat. No. 7,838,660; Ser. No. 12/818,016; Ser. No. 13/394,649). In the Ser. No. 12/818,016 patent application (“the '016 application”), entitled “Th-1 associated microRNAs and their use for tumor immunotherapy,” Okada et al. show that transgenic expression of the miR-17-92 cluster in T cells results in the enhanced infiltration/trafficking of CD3⁺VLA-4⁺cells to glioma sites. The '016 application claims a method of treating a subject with cancer through the transfection of isolated T cells with a heterologous nucleic acid molecule encoding the miR-17-92 transcript or a portion thereof, wherein the portion comprises the coding sequence for miR-17-3p, miR-18a, miR-19a, miR-20a, miR-19b-1 or miR-92a-1. The disclosure further anticipates that Tc1 cells transfected with miR-17-19b will demonstrate higher proliferation levels compared to control.

The miR-17-92 cluster encodes for 7 miRs: miR-17-5p, miR-17-3p, miR-18a, miR-19a, miR20a, miR-19b-1, and miR-92a-1 (Okada et al., Int J Biochem Cell Biol. (2010), 42(8):1256-61). Although the '016 specification states that “in some embodiments, the Th1-associated miR is selected from . . . miR-155,” the disclosure provides no expectation of success with regard to miR-155. The '016 specification does not show retroviral transductions of T cells with a nucleic acid molecule encoding a miR-155 transcript, the effect of such transductions on the T cells, nor does it show enhanced expansion of such cells once reintroduced into a subject with cancer. Thus, the present invention is advantageous over the prior art.

Use of miR-155 for the Enhancement of Anti-Viral and Anti-Bacterial Immunity.

In some embodiments of the present invention, the disease state is a viral infection. Although the treatment of influenza A is exemplified herein, any type of viral infection can be treated using the present invention. Viral infections caused by RNA or DNA viruses can be treated. In one example, the viral infection is caused by influenza A. In another example, the viral infection is caused by HIV.

The use of miRs as anti-viral therapeutics is known in the art (see for example Ser. No. 13/394,649; Ser. No. 13/262,086). In the Ser. No. 13/262,086 patent application (“the '086 application”), Buck claims a method of modulating host cell miRs, through the use of an antiviral compound, to inhibit viral propagation and/or replication in those host cells. Specifically, the '086 application claim a method of treating a subject suffering from one or more viral infections, diseases and/or conditions comprising administering a pharmaceutically effective amount of multi-species antiviral compound capable of modulating or mimicking the expression, function and/or activity of one or more host cell miRs.

In the Ser. No. 13/394,649 patent application (“the '649 application”), entitled “Method for the preparation of micro-RNA and its therapeutic application,” Velin et al., claim a method of treating a disease through the administration of a composition comprising a therapeutically effective amount of miR. In the '649 application, the therapeutically effective amount of miR is upregulated in a body fluid or element thereof upon activation of said body fluid. According to the disclosure, activation of the body fluid involves treatment of the body fluid with a surface that is able to trigger an immunological response.

Neither the '086 nor the '649 applications teach enhanced T cell mediated immunity resulting from increased expansion of T cells upon transduction of those cells with a nucleic acid molecule encoding a miR-155 transcript. Thus, the present invention is advantageous over the prior art.

In some embodiments of the present invention, the disease state is a bacterial infection. Although the treatment of Listeria monocytogenes is exemplified herein, any type of bacterial infection can be treated using the present invention.

Use of miR-155 to Enhance T Cell Expansion.

The present invention provides methods of enhancing T cell expansion in a subject having a disease state. In some embodiments, a nucleic acid molecule encoding a miR-155 transcript can be introduced into tumor-specific CD8+ T cells from patients, which are reintroduced into the patient. In some embodiments, a nucleic acid molecule encoding a CAR and a miR-155 transcript can be introduced into tumor-specific T cells from patients, which are reintroduced into the patient. In other embodiments, a nucleic acid molecule encoding a CAR and a nucleic acid molecule encoding a miR-155 transcript can be introduced into subject-specific T cells.

Expansion as used herein refers to increased number of cells. Thus, expansion encompasses increased proliferation of T cells and/or inhibition of T cell death.

### EXAMPLES

### Example 1

### CD8+ T Cells Markedly Upregulate miR-155 Expression Upon In Vitro Activation and In Vivo During Infection

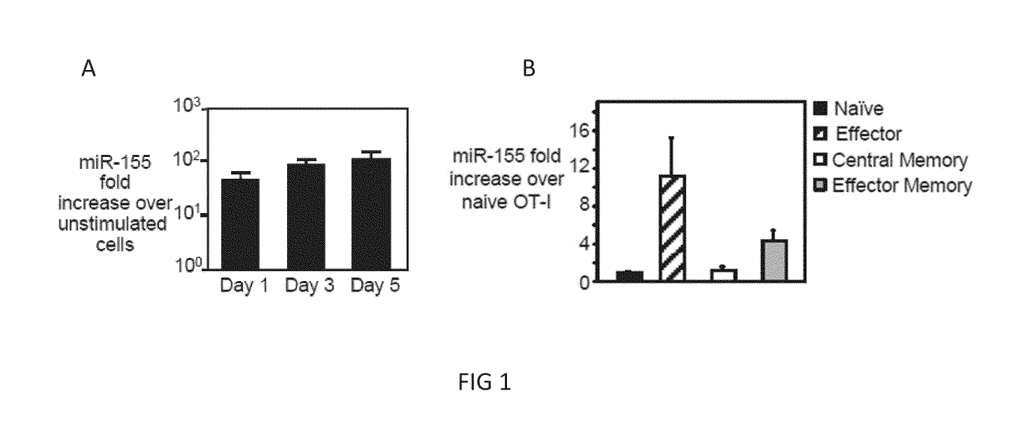

Upon in vitro stimulation, naïve CD8+ T cells rapidly increase miR-155 mRNA expression. Activation of purified CD8+ T cells with solid phase anti-CD3/anti-CD28 antibodies for 24 h resulted in a 42-fold increase of miR-155 compared to naïve, unstimulated CD8+ T cells. On days 3 and 5 of activation, the levels of miR-155 further increased to 83- and 104-fold, respectively, over naïve unstimulated controls. (FIG. 1A).

To determine if miR-155 is also expressed in vivo during CD8+ T cell responses, miR-155 was measured in sorted donor OT-I CD8+ T cells isolated from congenic Thy1.2+ mice that had been adoptively transferred with Thy1.1 OVA(257-264)-specific TCR-transgenic OT-I cells, and then infected with the OVA(257-264) peptide expressing WSN-OVA influenza virus. Donor lung day 10 effector CD44+CD62L− OT-I cells were found to express 11-fold more miR-155 relative to naïve CD44−CD62L+ OT-I cells (FIG. 1B). In contrast, donor day 60 splenic central memory CD44+CD62L+ OT-I cells downregulated miR-155 to naïve cell levels (1.2-fold relative to naïve CD8+ T cells. The donor day 60 splenic effector memory CD44+CD62L− OT-I cell subset showed a 4.4-fold increase in miR-155 levels that was intermediate between primary effector and central memory cells. The sustained induction of miR-155 expression seen in in vitro and ex vivo CD8+ T cells demonstrates that miR-155 may play a role in regulating CD8+ T cell responses.

### Example 2

### miR-155 is Required for the Generation of an Optimal CD8+ T Cell Response to Influenza Virus Infection

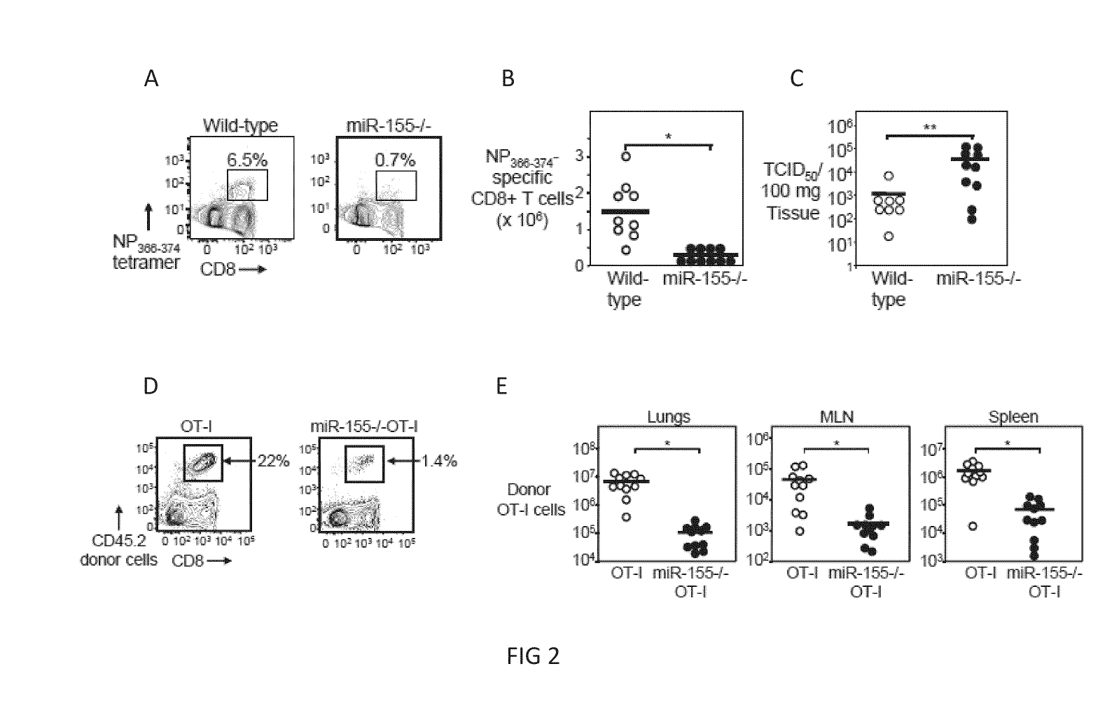

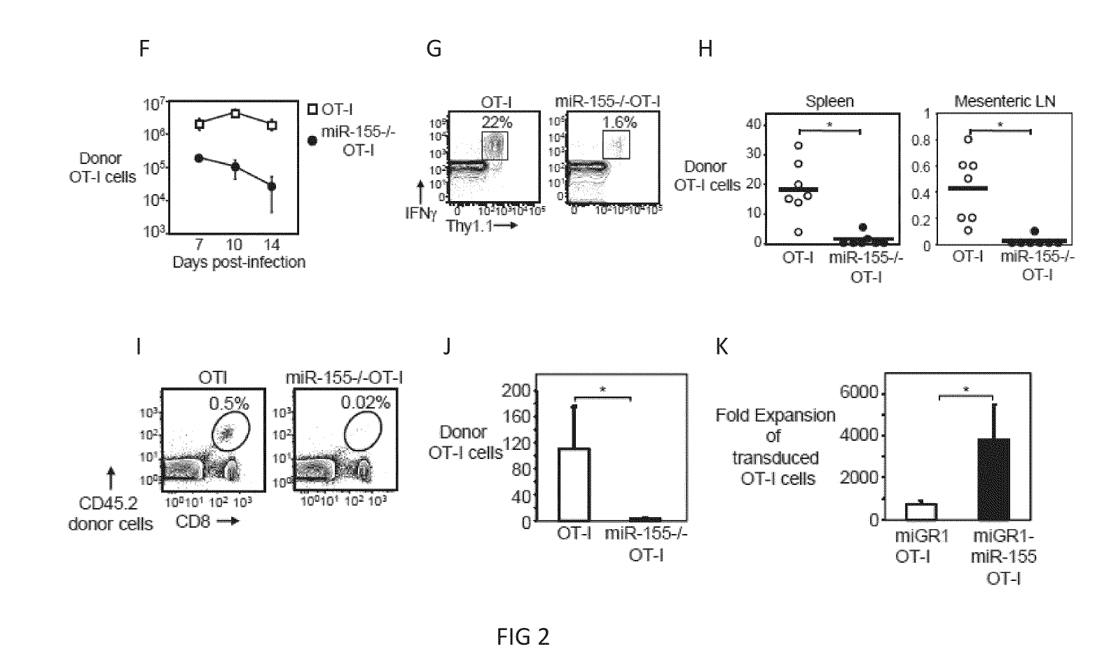

To test whether miR-155 plays a role in CD8+ T cell responses in vivo, miR-155−/− and congenic wild-type C57BL/6 mice were infected with a sublethal dose of A/PR/8/34 influenza virus. Antigen-specific CD8+ T cells were identified by surface staining with MHC class I tetramers loaded with the NP(366-374) immunodominant peptide. miR-155−/− mice showed greatly reduced frequencies and numbers of peak day 10 lung NP(366-374)-specific CD8+ T cells compared to wild-type mice with numbers of pulmonary NP(366-374)-specific CD8+ T cells in miR-155−/− mice reduced by 6-fold compared to wild-type animals (FIGS. 2A and 2B). This reduction of NP(366-374)-specific CD8+ T cells was also observed in mediastinal lymph nodes (MLN) and spleens (data not shown). This reduced CD8+ T cell response in miR-155−/− mice was accompanied by impaired viral clearance (FIG. 2C).

### Example 3

### miR-155 Deficiency Confers an Intrinsic Defect in CD8+ T Cells During Primary Responses to Viral and Bacterial Infections

Since multiple immune cells are known to be affected by miR-155, we sought to determine whether miR-155 deficiency conferred an intrinsic defect in the CD8+ T cell ability to respond to pathogens. CD45.2+ miR-155−/−OT-I mice (on a C57BL/6 background) were generated and 10⁴ CD45.2+ miR-155−/−OT-I or wild-type CD45.2+OT-I CD8+ T cells were adoptively transferred into congenic CD45.1+ wild-type mice that were infected with influenza virus WSN-OVA. At day 10 post-infection, donor lung OVA(257-264)-specific CD8+ T cells were reduced 62-fold in hosts transferred with miR-155−/−OT-I cells compared to recipients of OT-I cells (FIG. 2D and FIG. 2E). This reduction was also found in MLN and spleens (FIG. 2E), indicating that survival and/or proliferation but not trafficking of CD8+ T cells was affected by miR-155 deficiency. The reduced peak response of miR-155−/−OT-I cell was not due to a shift in the kinetics (FIG. 2F).

To examine whether miR-155 deficiency affected CD8+ T cell responses to bacterial infection, Thy1.1+ OT-I or Thy1.1+ miR-155−/−OT-1 cells were adoptively transferred into Thy1.2+ congenic mice which were infected with ovalbumin-expressing Listeria monocytogenes (L.m.-OVA). Mice transferred with miR-155−/−OT-I CD8+ T cells had 15-fold lower numbers of donor OVA(257-264)-specific CD8+ T cells on day 7 postinfection in spleens compared to OT-I cell recipients (FIGS. 2G and 2H). Similar reductions were observed in the mesenteric lymph nodes (FIG. 2H). These results demonstrate that miR-155 plays an intrinsic role in regulating primary CD8+ T cell responses against viral and bacterial pathogens.

### Example 4

### miR-155 is Required for Generation of CD8+ T Cell Memory

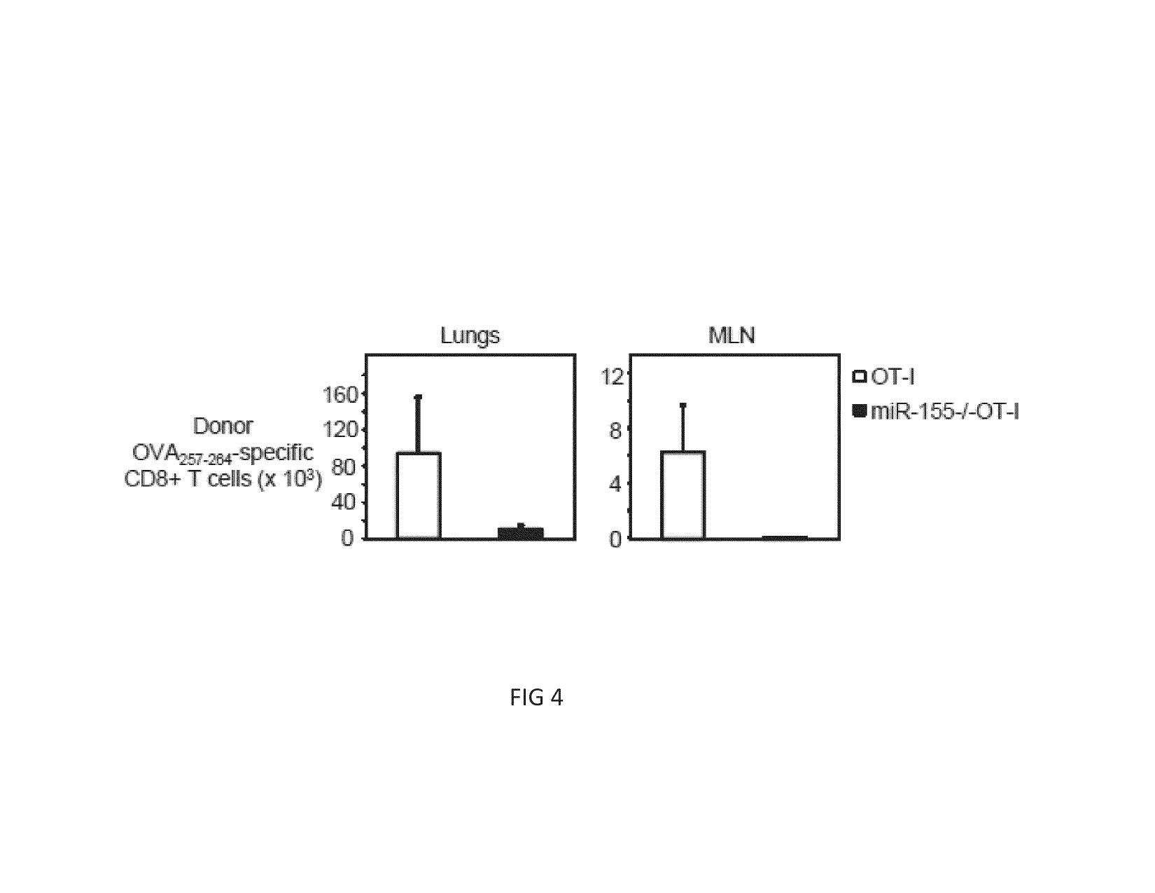

To evaluate miR-155's role in the generation of memory CD8+ T cells, 10⁴ CD45.2+ miR-155−/−OT-I or CD45.2+ OT-I cells were adoptively transferred into CD45.1+mice that were infected with influenza virus WSN-OVA. At day 60 post-infection, donor memory miR-155−/−OT-I cells were reduced by 37-fold in the spleens relative to the wild-type OT-I (FIGS. 2I and 2J). This reduction in donor memory miR-155−/− OT-I cells was also apparent in the MLNs and the lungs (FIG. 4). These findings indicate that miR-155 is required for the generation of a CD8+ T cell memory pool during pathogenic infections.

### Example 5

### Overexpression of miR-155 Augments CD8+ T Cell Responses

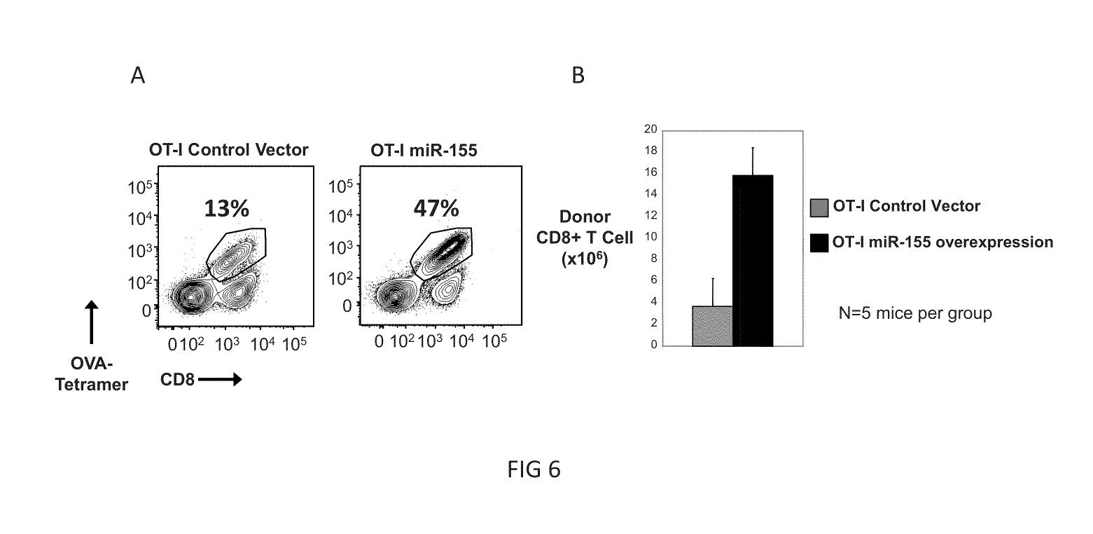

Since miR-155 deficiency inhibited CD8+ T cell responses, we investigated whether miR-155 overexpression would enhance CD8+ T cell immunity. Thy1.1+OT-I CD8+ T cells were ex vivo transduced with a miR-155-expressing MIGR1 retroviral vector or a MIGR1 control vector that co-expresses GFP. After 48 hours, 10² donor GFP+ OT-I CD8+ T cells were intravenously transferred into Thy1.2+C57BL/6 mice, and recipients were infected with influenza virus WSN-OVA. At 10 days post-infection pulmonary miR-155-expressing MIGR1 transduced OT-I cells expanded five times more (3845±1702 fold expansion over transferred cell number) compared to the control vector MIGR1 transduced OT-I cells (766±164 fold expansion) (FIG. 2K). Similar increases were also observed in the MLNs and the spleens (data not shown). The increased expansion of virus-specific CD8+ T cells was also demonstrated with transfers of larger numbers of miR-155 overexpressing CD8+ T cells. When 10⁴ OT-I CD8+ T cells were transduced with a miR-155-expressing retroviral vector and intravenously transferred into C57BL/6 mice, and then recipients were infected with WSN-OVA influenza virus, we again observed large expansions of OT-I cells expressing miR-155 compared to control retrovirus transduced OT-I. The 10 days post-infection pulmonary miR-155-expressing transduced OT-I cells expansion is shown in FIG. 6. Thus overexpression of miR-155 augments CD8+ T cell responses.

### Example 6

### miR-155 Regulates the Proliferation of CD8+ T Cells

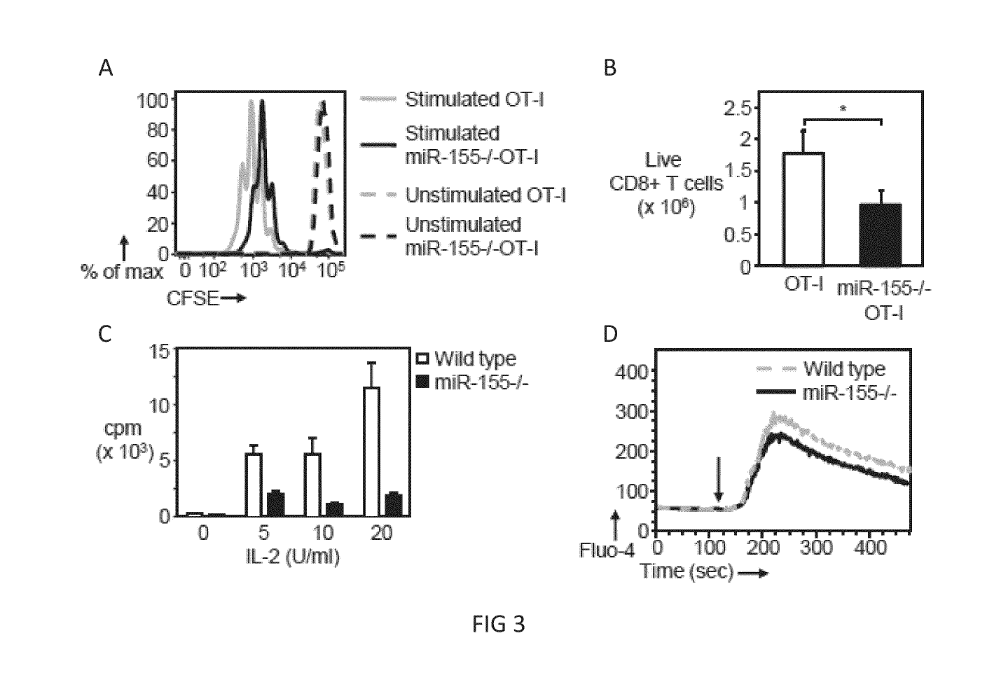

To determine whether the reduced in vivo responses of miR-155−/− CD8+ T cells were due to impaired proliferation, splenic miR-155−/−OT-I or wild-type OT-I cells were purified, labeled with carboxy fluorescein diacetate, succinimidyl ester (CF SE) and stimulated with OVA(257-264)—pulsed irradiated splenocytes and 10 U/ml IL-2. After four days, miR-155−/−OT-I cells displayed reduced proliferation with fewer divisions relative to control OT-I cells (FIG. 3A) and this was accompanied by a 2-fold reduction in cell number of miR-155−/−OT-I CD8+ T cells, when compared to wild-type OT-I CD8+ T cells (FIG. 3B). A proliferative defect of miR-155−/− CD8+ T cells was also found following stimulation with solid phase anti-CD3 antibody plus IL-2 stimulation. Compared to wild-type CD8+ T cells, miR-155−/− CD8+ T cells exhibited reduced ³H-thymidine incorporation (FIG. 3C). MiR-155−/− CD8+ T cells showed no significant increase in spontaneous, CD95-induced apoptosis and activation-induced cell death (AICD) (data not shown). Since miR-155 can regulate cytokine production, we also examined in vitro IL-2, IFNγ, TNFα, IL-4 and IL-5 production and in vivo IFNγ and TNFα expression and found no difference between miR-155−/− and wild type CD8+ T cells (data not shown). The above suggest that defects in proliferative capacity are responsible for the reduced miR-155−/− CD8+ T cell responses.

### Example 7

### miR-155 Overexpression in CD8+ T Cells Increases their Anti-Tumor Activity Against Established Tumors

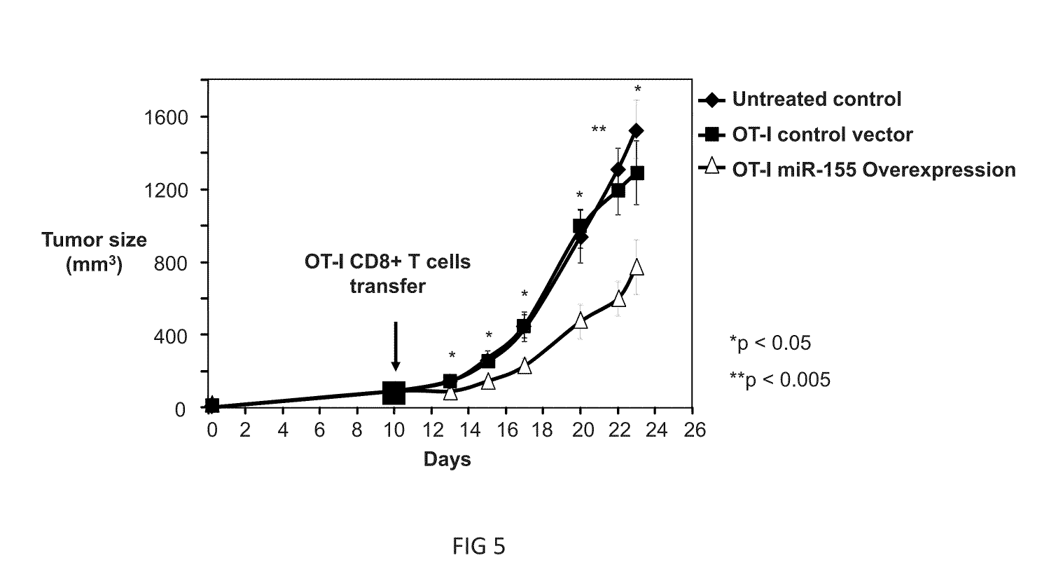

To test the anti-tumor effect of overexpressing miR-155 in CD8+ T cells in established tumors, we injected C57BL/6 mice in the flank with the AE17 OVA-expressing mesothelioma tumor (AE17.OVA) and allowed the tumors to grow for 10 days. On day 10, we intravenously transferred into the mice 2×10⁵ OT-I cells (TCR-transgenic OVA-specific CD8+ T cells) that were transduced with a miR-155-expressing retroviral vector or a control vector. When tumors were measured daily, we found that miR-155-expressing OT-I cells controlled tumors much better than the control retrovirus transduced OT-I cell that showed no effect (FIG. 5). This data directly demonstrates that miR-155 overexpression in CD8+ T cells can enhance their activity against established tumors.

### Example 8

### CD8+ T Cells Overexpressing miR-155 for Anti-Cancer Immunity

This example demonstrates the use of enhanced miR-155 expression in T cells as a means of providing a subject with anti-cancer immunity.

The anti-cancer immunity enhancing effect of miR-155 can be tested by utilizing immunodeficient mice, such as Rag2−/−γc−/− double knockout mice, among others, which can be implanted with human tumors without these tumors being rejected. T cells isolated from either patient tumors or peripheral blood can be transduced or transfected to express miR-155 alone or miR-155 in combination with a chimeric antigen receptor (CAR). The CAR and miR-155 can be introduced into the T cells with a single nucleic acid molecule encoding the CAR and miR-155 transcript. Alternatively, nucleic acid molecules encoding CAR and the miR-155 transcript can be introduced with separate nucleic acid molecules. Expression of CAR and miR-155 can be achieved by lentiviral, retroviral or other viral transduction or DNA or RNA transfection. CD8+ or CD4+ T cells can be expanded with anti-CD3+anti-CD28 antibodies in vitro either before or after the introduction of CAR and miR-155 into the cells. In some cases anti-4-1BB antibodies can be used for expansion. Once sufficient numbers of T cells expressing CAR and/or miR-155 are generated, they can be introduced either intravenously or intraperitonealy into the tumor-bearing immunodeficient mouse. This adoptive transfer of CAR and miR-155 expressing T cells will result in enhanced expansion of the T cells, leading to enhanced tumor killing and elimination or reduction of tumor burden in mice.

This method can be adapted for use in other subjects such as humans.

The disclosures of each patent, patent application, and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed:

1. A method of increasing CD8+ T cell mediated immunity in a subject having a disease state comprising:
isolating a population of the subject's CD8+ T cells;
introducing a nucleic acid molecule encoding a miR-155 transcript into the isolated CD8+ T cells; and
reintroducing the CD8+ T cells into said subject.

2. The method of claim 1, wherein the nucleic acid molecule is introduced into the isolated CD8+ T cells by transduction or transfection.

3. The method of claim 1, wherein the nucleic acid molecule encoding the miR-155 transcript is the BIC gene or a portion thereof.

4. The method of claim 1, wherein the disease state is cancer.

5. The method of claim 1, wherein the disease state is a viral infection.

6. The method of claim 1, wherein the diseased state is a bacterial infection.

7. The method of claim 1, wherein the CD8+ T cells are isolated from the blood of the subject.

8. The method of claim 1, wherein the CD8+ T cells are isolated from a solid tumor from said subject.

9. The method of claim 1, wherein the nucleic acid molecule encoding a miR-155 transcript is encoded within an expression vector.

10. A method of increasing T cell mediated immunity in a subject having a disease state comprising:
isolating a population of the subject's T cells;
introducing a nucleic acid molecule encoding a chimeric antigen receptor and a miR-155 transcript into the isolated T cells; and
reintroducing the T cells into said subject.

11. The method of claim 10, wherein the population of T cells comprises both CD4+ T cells and CD8+ T cells

12. The method of claim 10, wherein the population of T cells comprises either CD4+ T cells or CD8+ T cells.

14. The method of claim 10, wherein the nucleic acid molecule is introduced into the isolated T cells by transduction or transfection.

15. The method of claim 10, wherein the nucleic acid molecule encoding the miR-155 transcript is the BIC gene or a portion thereof.

16. The method of claim 10, wherein the disease state is cancer.

17. The method of claim 10, wherein the disease state is a viral infection.

18. The method of claim 10, wherein the diseased state is a bacterial infection.

19. The method of claim 10, wherein the T cells are isolated from the blood of the subject.

20. The method of claim 10, wherein the T cells are isolated from a solid tumor from said subject.

21. The method of claim 10, wherein the nucleic acid molecule encoding a chimeric antigen receptor and the miR-155 transcript is encoded within an expression vector.

22. The method of claim 10, wherein a nucleic acid molecule encoding miR-155 is located within the intracellular tail of the chimeric T cell receptor.

23. The method of claim 22, wherein a nucleic acid molecule encoding miR-155 alone is located within the intracellular tail of the chimeric T cell receptor.

24. The method of claim 22, wherein a nucleic acid molecule encoding miR-155 together with a costimulatory sequence is located within the intracellular tail of the chimeric T cell receptor.

25. A method of increasing T cell mediated immunity in a subject having a disease state comprising:
isolating a population of the subject's T cells;
introducing a nucleic acid molecule encoding a chimeric antigen receptor into the T cells;
additionally introducing a nucleic acid molecule encoding a miR-155 transcript in the T cells; and
reintroducing the T cells into said subject.

26. The method of claim 25, wherein the population of T cells comprises both CD4+ T cells and CD8+ T cells.

27. The method of claim 25, wherein the population of T cells comprises either CD4+ T cells or CD8+ T cells.

28. The method of claim 25, wherein the nucleic acid molecule is introduced into the isolated T cells by transduction or transfection.

29. The method of claim 25, wherein the nucleic acid molecule encoding the miR-155 transcript is the BIC gene or a portion thereof.

30. The method of claim 25, wherein the disease state is cancer.

31. The method of claim 25, wherein the disease state is a viral infection.

32. The method of claim 25, wherein the diseased state is a bacterial infection.

33. The method of claim 25, wherein the T cells are isolated from the blood of the subject.

34. The method of claim 25, wherein the T cells are isolated from a solid tumor from said subject.

35. The method of claim 25, wherein the nucleic acid molecule encoding the chimeric antigen receptor and the nucleic acid molecule encoding the miR-155 transcript are encoded within expression vectors.
